(19) United States
(12) Patent Application Publication
(10) Pub. No.: US 2020/0179271 A1
(43) Pub. Date: Jun. 11, 2020

(54) COMPOSITIONS AND METHODS FOR TREATING ACUTE CANNABINOID OVERDOSE WITH A CANNABINOID RECEPTOR ANTAGONIST

(71) Applicant: Opiant Pharmaceuticals, Inc., Santa Monica, CA (US)

(72) Inventor: Phil SKOLNICK, Potomac, MD (US)

(21) Appl. No.: 16/704,699

(22) Filed: Dec. 5, 2019

Related U.S. Application Data
(60) Provisional application No. 62/776,888, filed on Dec. 7, 2018.
(60) Provisional application No. 62/862,832, filed on Jun. 18, 2019.

(51) Int. Cl. A61K 9/00; A61K 31/397

(52) U.S. Cl. CPC A61K 9/0019; A61K 31/397

(57) ABSTRACT

Disclosed herein are formulations and methods for reversing cannabinoid overdose or one or more symptoms thereof, comprising parenterally administering a CB1 antagonist in an amount sufficient to reverse the cannabinoid overdose or symptom(s).

This application claims the benefit of priority of U.S. Provisional Applications No. 62/776,888, filed on Dec. 7, 2018, and 62/862,832, filed Jun. 18, 2019, the disclosures of which are hereby incorporated by reference as if written herein in their entireties.

Consumption of both synthetic cannabinoids (SCs) and ΔA⁹-tetrahydrocannabinol (THC), a naturally occurring psychoactive compound found in cannabis, can have serious medical consequences leading to emergency department visits and hospitalization.

Synthetic cannabinoids (SCs) are a heterogeneous class of organic molecules that bear little structural similarity to THC (Shalit, et al., 2016). These compounds were initially synthesized as research tools to study the function of cannabinoid receptors (CR); reports of the illicit sale and misuse of SCs first appeared in the early 2000s. SCs are typically dissolved in solvents like acetone and ethanol and sprayed over oregano or similar plant materials. These are sold over the internet, in gas stations and other retail establishments in packets with names such as “K2,” “Spice,” and “Scooby Snax.” The packets, often labelled as incense, potpourri, or herbal blends, carry a disclaimer indicating the material is not for human consumption. In this form, SCs are generally smoked, and often mixed with tobacco or cannabis. More potent (see below) and often far less expensive than cannabis, SCs are often preferred because they would not register as a positive in a drug screen for cannabis.

SCs are generally far more potent than THC at CB1 receptors [those located primarily in the central nervous system and producing the most problematic side effects]: some SCs have 40-80-fold higher affinities than THC [Shalit, et al., 2016] and can be more efficacious (effective) at signaling through CB-1 receptors (Burkey, et al, 1997). This latter property of SCs results in more pronounced symptoms on overdose compared with THC (Zaurova, et al. 2016, J Med Toxicol 12; Winstock et al. 2015 J Psychopharmacol). While many of these SCs are now listed as Schedule I by the Drug Enforcement Administration, minor structural modification of this heterogeneous group of compounds can circumvent scheduling. Both cardiovascular (hypertension, tachycardia) and neuropsychiatric symptoms (agitation, confusion, drowsiness, hallucinations) are often seen. Nausea and vomiting have also been reported in 18-25% of patients presenting to an emergency department with SC intoxication (Rowley, et al. Amer. J. Emerg Med. 2017). In one recent study, it was estimated that over 20% of patients seen in an emergency department for SC poisoning were hospitalized for either medical or psychiatric treatment, and 5% were admitted to intensive care (Rowley, et al., 2016).

Excessive consumption of Δ⁹-tetrahydrocannabinol (THC), a naturally occurring psychoactive compound found in marijuana, can also lead to overdose, producing a similar spectrum of symptoms resulting in emergency room visits and hospitalization (Cao, et al., 2016 Clinical Tox; Bui et al., 2015, West J. Emerg Med). This scenario most often occurs following ingestion of edible marijuana products that are processed as brownies, cookies and candies (e.g. chocolates, fruit slices), and beverages. THC content is often very high in these products and consuming an entire edible (e.g. a brownie) intended as a multi-dose product can result in overdose. Furthermore, the absorption of THC is slow following oral administration (maximum plasma concentrations are estimated to be attained 60-300 min following ingestion of a cookie containing 20 mg of THC) [Newmeyer, et al., 2017 Clinical Chemistry], and this delay relative to smoked cannabis often leads to an overdose if an individual consumes multiple edibles because the initial dose ‘isn't working’ (Cao, et al., 2016). Children are particularly vulnerable to THC overdose resulting from consumption of edibles that are frequently packaged as cookies, candies, and brownies. Lethargy and ataxia are the most common signs of THC overdose reported in the emergency department, and often results in hospitalization. In one study (Richards, et al., J. Pediatr. 190, 2017), 18% of children hospitalized were admitted to a pediatric intensive care unit. Unintentional THC ingestion by children is well-documented, and has been characterized as a serious public health concern (Richards, et al.). Unintentional THC overdose by both adults and children continues to increase as more states legalize the recreational use of cannabis. Thus, in a 2016 analysis of the National Poison Data System, Cao, et al (2016) reported that the highest number of calls to US poison centers were from Colorado and Washington, two states that have legalized the recreational use of cannabis. Further, during the two year duration of this study (2013-2015), the number of calls increased every year, a trend consistent with the increasing number of emergency department visits reported between 2004-2011 (J ADDIC MED. 2016: Zhu and Wu).

At present, there are no medications approved to treat acute cannabinoid overdose. Individuals treated by emergency medical services or admitted to the emergency department receive supportive therapy, often consisting of IV fluids and sedatives. Supplemental oxygen, restraints and intubation are also used, albeit with less frequency (Rowley et al., Amer. J. of Emerg. Med. 35:1506-1509, 2017). Moreover, significant emergency department resources are used in treating cannabinoid overdose; the costs associated with hospital visits related to cannabinoid poisoning are, on average, $4500 (Rowley, et al., 2017). Thus, there is a clear need for rapid acting, specific medications to reverse the symptoms of overdose produced by both synthetic cannabinoids and THC.

The identification of cannabinoid receptors more than 25 years ago (Matsuda, et al. Nature, 1990) led to the synthesis of multiple chemicals which bind to these receptors with high affinity and specificity. In addition to SCs described above, multiple compounds have been synthesized that, on binding to the CB-1 receptor, function as antagonists. That is, unlike SCs and THC, these compounds do not activate CB-1 receptor mediated signaling but can block the effects of both endogenous and exogenous cannabinoids (Janero and Makryiannis, Expert Opinion on Emerging Drugs, 2009). Multiple, structurally diverse high affinity CB-1 antagonists have been described in the peer reviewed literature (Janero and Makryiannis). Several of these compounds have been examined in clinical studies for indications as diverse as smoking cessation, obesity, and schizophrenia (Janero and Makryiannis). There are two reports in the clinical literature that oral administration of CB-1 antagonists (surinabant, drinabant) can prevent (block) some of the pharmacological effects of smoked or inhaled THC in normal volunteers (Klumpers, et al., 2012; Zuurman, et al., 2010). However, both the slow onset of these compounds following oral administration (maximum plasma concentrations achieved in 2-3 h) and the range of symptoms (that can range from extreme lethargy to highly agitated) make oral dosing impractical. Moreover, a useful, effective antidote must be able to rapidly reverse (that is, be administered after the cannabinoid) the most prominent and disturbing symptoms of cannabinoid overdose.

Accordingly, described herein are compositions and methods for reversing the symptoms of cannabinoid overdose using the CB-1 antagonist drinabant (AVE 1625) or a salt or polymorph thereof via parenteral administration.

### DETAILED DESCRIPTION

Disclosed herein are compositions and methods for reversing cannabinoid overdose and/or one or more symptoms thereof comprising (parenterally administering) a CB1 antagonist in an amount sufficient to reverse the cannabinoid overdose and/or symptom(s).

In certain embodiments, the CB-1 antagonist is drinabant, or a salt or polymorph thereof.

In one embodiment, provided herein is a composition formulated for parenteral administration comprising an amount of drinabant or a salt or polymorph thereof effective to reverse cannabinoid overdose, or one or more symptoms thereof in a subject (Embodiment 1).

In another embodiment, provided herein is a method of reversing one or more symptoms cannabinoid overdose comprising parenterally administering the CB-1 antagonist drinabant or a salt or polymorph thereof, preferably in an amount sufficient to reverse the cannabinoid overdose and/or symptom(s) (Embodiment 2).

In certain embodiments (e.g., either of Embodiments 1 and 2), the cannabinoid overdose is acute cannabinoid overdose (ACO) (Embodiment 3).

In certain embodiments (e.g., any of Embodiments 1-3), the plasma concentration of drinabant sufficient to reverse the cannabinoid overdose symptom(s) ranges from 200 to about 730 ng/ml (Embodiment 4).

In certain embodiments (e.g., any of Embodiments 1-4), such as, for example, in an emergency setting, the parenteral route of administration is chosen from among intravenous (IV), intramuscular (IM), and subcutaneous (SC) (Embodiment 5).

In certain embodiments (e.g., any of Embodiments 1-5), the amount of drinabant, or a salt or polymorph thereof. sufficient to reverse the cannabinoid overdose symptom(s) is between about 1 mg and about 150 mg per dose, between about 1 mg and about 100 mg per dose, between about 1 mg and about 60 mg per dose, between about 15 mg and about 60 mg per dose, between about 30 mg and about 60 mg per dose, between about 1 mg and about 30 mg per dose, or between about 15 mg and about 30 mg per dose (Embodiment 6).

In certain embodiments (e.g., either of Embodiments 5 and 6), the parenteral route of administration is IV (Embodiment 7).

In certain embodiments (e.g., Embodiment 7), the amount of drinabant, or a salt or polymorph thereof. sufficient to reverse the cannabinoid overdose or symptom(s) thereof is between about 1 mg and about 60 mg per IV dose (Embodiment 8).

In certain embodiments (e.g., Embodiment 8), the amount of drinabant, or a salt or polymorph thereof. sufficient to reverse the cannabinoid overdose or symptom(s) thereof is between about 15 mg and about 60 mg per IV dose (Embodiment 9).

In certain embodiments (e.g., Embodiment 8), the amount of drinabant, or a salt or polymorph thereof. sufficient to reverse the cannabinoid overdose or symptom(s) thereof is between about 15 mg and about 30 mg per IV dose (Embodiment 10).

In certain embodiments (e.g., Embodiment 8), the amount of drinabant, or a salt or polymorph thereof. sufficient to reverse the cannabinoid overdose or symptom(s) thereof is between about 30 mg and about 60 mg per IV dose (Embodiment 11).

In certain embodiments (e.g., any of Embodiments 7-11), the IV dose is delivered by IV injection (Embodiment 12).

In certain embodiments (e.g., Embodiment 12), the IV dose is delivered in a liquid volume of between about 1 and about 20 mL (Embodiment 13).

In certain embodiments (e.g., Embodiment 13), the IV dose is delivered in a liquid volume of between about 1 and about 20 mL, distributed across two or more pushes (Embodiment 14).

In certain embodiments (e.g., any of Embodiments 7-11), the IV dose is delivered by IV infusion (IVN) (Embodiment 15).

In certain embodiments (e.g., Embodiment 15), the IVN dose is delivered in a liquid volume of about 50 mL to about 500 mL, about 50 mL to about 125 mL, or about 250 mL to about 500 mL (Embodiment 16). In certain embodiments (e.g., Embodiment 15), the IVN dose is delivered in a liquid volume of about 50 mL, about 100 mL, about 125 mL, about 250 mL, or about 500 mL (Embodiment 17).

In certain embodiments (e.g., any of Embodiments 15-17), the IVN dose is delivered over a period of about 30 min to about 120 min (Embodiment 18). In certain embodiments (e.g., any of Embodiments 15-17), the IVN dose is delivered over a period of about 30 min, about 60 min, about 90 min, or about 120 min (Embodiment 19).

In certain embodiments, the IVN dose is delivered in a liquid volume of between about 125 to about 500 mL (Embodiment 20).

In certain embodiments (e.g., any of Embodiments 15-20), the IVN dose is delivered over a period of about 1 hour to about 2 hours (Embodiment 21).

In certain embodiments (e.g., any of Embodiments 15-21), the IVN dose is delivered at a rate of about 0.5 mL/min to about 2 mL/min (Embodiment 22).

In certain embodiments (e.g., either of Embodiments 5 and 6), the parenteral route of administration is IM or SC (Embodiment 23).

In certain embodiments (e.g., Embodiment 23), the amount of drinabant, or a salt or polymorph thereof sufficient to reverse the cannabinoid overdose symptom(s) is between about 5 mg and about 100 mg per IM dose (Embodiment 24).

In certain embodiments (e.g., Embodiment 24), the amount of drinabant, or a salt or polymorph thereof sufficient to reverse the cannabinoid overdose symptom(s) is between about 5 mg and about 60 mg per IM dose (Embodiment 25).

In certain embodiments (e.g., Embodiment 24), the amount of drinabant, or a salt or polymorph thereof sufficient to treat, reverse, or reduce the cannabinoid overdose or one or more symptoms thereof is between about 5 mg and about 30 mg per IM dose (Embodiment 26).

In certain embodiments (e.g., Embodiment 24), the amount of drinabant, or a salt or polymorph thereof sufficient to treat, reverse, or reduce the CHS or one or more symptoms thereof is between about 30 mg and about 60 mg per IM dose (Embodiment 27).

In certain embodiments (e.g., any of Embodiments 24-27), the IM dose is delivered in a liquid volume of up to about 2.5 ml (Embodiment 28). In certain embodiments (e.g., Embodiment 28), the IM dose is delivered in a liquid volume of about 1 mL to about 2.5 ml (Embodiment 29).

In certain embodiments (e.g., any of Embodiments 24-29), the IM dose is delivered by injection into a deltoid or gluteal muscle (Embodiment 30).

In certain embodiments (e.g., Embodiment 23), the amount of drinabant, or a salt or polymorph thereof sufficient to reverse the cannabinoid overdose symptom(s) is between about 5 mg and about 100 mg per SC dose (Embodiment 31).

In certain embodiments (e.g., Embodiment 31), the amount of drinabant, or a salt or polymorph thereof sufficient to reverse the cannabinoid overdose symptom(s) is between about 5 mg and about 60 mg per SC dose (Embodiment 32).

In certain embodiments (e.g., Embodiment 31), the amount of drinabant, or a salt or polymorph thereof sufficient to treat, reverse, or reduce the cannabinoid overdose or one or more symptoms thereof is between about 5 mg and about 30 mg per IM dose (Embodiment 33).

In certain embodiments (e.g., Embodiment 31), the amount of drinabant, or a salt or polymorph thereof sufficient to treat, reverse, or reduce the CHS or one or more symptoms thereof is between about 15 mg and about 60 mg per IM dose (Embodiment 34).

In certain embodiments (e.g., Embodiment 31), the amount of drinabant, or a salt or polymorph thereof sufficient to treat, reverse, or reduce the CHS or one or more symptoms thereof is between about 15 mg and about 30 mg per IM dose (Embodiment 35).

In certain embodiments (e.g., Embodiment 31), the amount of drinabant, or a salt or polymorph thereof sufficient to treat, reverse, or reduce the CHS or one or more symptoms thereof is between about 30 mg and about 60 mg per IM dose (Embodiment 36).

In certain embodiments (e.g., any of Embodiments 31-36), the SC dose is delivered in a liquid volume of up to about 1.5 ml (Embodiment 37). In certain embodiments (e.g., Embodiment 37), the SC dose is delivered in a liquid volume of about 1 mL to about 1.5 ml (Embodiment 38).

In certain embodiments (e.g., any of Embodiments 5-38), the IV, IVN, IM, or SC dose or injection contains at least one agent that acts as a non-ionic solubilizer and/or emulsifying agent (Embodiment 39).

In certain embodiments (e.g., Embodiment 39), the agent that acts as a non-ionic solubilizer and/or emulsifying agent comprises polyoxyl 15 hydroxystearate or a mixture of polyglycol mono- and di-esters of 12-hydroxystearic acid (Embodiment 40).

In certain embodiments (e.g., Embodiment 40), the agent that acts as a non-ionic solubilizer and/or emulsifying agent which comprises polyoxyl 15 hydroxystearate is Kolliphor® HS 15 (CAS No. 70142-34-6) or Solutol HS 15 (polyglycol mono- and di-esters of 12-hydroxystearic acid with about 30% polyethylene glycol) (Embodiment 41).

In certain embodiments (e.g., any of Embodiments 1-41), the symptom(s) of cannabinoid overdose is/are chosen from cardiovascular symptom(s), neuropsychiatric symptom(s), and gastrointestinal symptom(s) (Embodiment 42).

In certain embodiments (e.g., Embodiment 42), the cardiovascular symptom(s) is/are chosen from hypertension and tachycardia (Embodiment 43).

In certain embodiments (e.g., Embodiment 42), the neuropsychiatric symptom(s) is/are chosen from agitation, confusion, drowsiness/lack of alertness, hallucinations, and feeling “high” (Embodiment 44).

In certain embodiments (e.g., Embodiment 42), the gastrointestinal symptom(s) is/are chosen from nausea and vomiting (Embodiment 45).

In certain embodiments (e.g., any of Embodiments 6-14 and 39-45), the onset of reversal of symptom(s) of cannabinoid overdose are apparent within 5-30 minutes following IV injection of drinabant, or a salt or polymorph thereof (Embodiment 46).

In certain embodiments (e.g., any of Embodiments 6-11, 15-22, and 39-45), the onset of reversal of symptom(s) of cannabinoid overdose are apparent within 30-60 minutes following IVN injection of drinabant, or a salt or polymorph thereof (Embodiment 47).

In certain embodiments (e.g., any of Embodiments 23-45), the onset of reversal of symptom(s) of cannabinoid overdose are apparent within about 15 min to about 45 min following IM or SC administration of drinabant, or a salt or polymorph thereof (Embodiment 48).

In certain embodiments (e.g., any of Embodiments 1-48), if no response is observed within about 30 min to about 45 min of a first administration of drinabant, and the presence of cannabinoids is confirmed or strongly suspected, a second dose may be administered (Embodiment 49).

### Definitions

When ranges of values are disclosed, and the notation “from n₁ . . . to n₂” or “between n₁ . . . and n₂” is used, where n₁ and n₂ are numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 mL (milliliters),” which is intended to include 1 mL, 3 mL, and everything in between to any number of significant figures (e.g., 1.255 mL, 2.1 mL, 2.9999 mL, etc.).

As used herein, the term “about” is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

As used herein, the term “agonist” refers to a moiety that interacts with, and activates, a receptor and thereby initiates a physiological or pharmacological response characteristic of that receptor.

As used herein, the term “antagonist” refers to a moiety that binds to a receptor, but which does not activate the intracellular response(s) initiated by the active form of the receptor when occupied by an agonist, and can thereby inhibit the intracellular responses that would be elicited by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist. It is noted that under certain conditions a compound can behave like an antagonist while under other conditions it can behave as an inverse agonist. Functionally, both an antagonist and an inverse agonist can block and/or reverse the effects of an agonist or partial agonist.

As used herein, reversal of symptom(s) of cannabinoid overdose is “apparent” when, in the judgment of a trained healthcare giver (e.g., physician, nurse practitioner, nurse, paramedic, or emergency medical technician), the symptom(s) have abated to a noticeable degree. Such a caregiver may use any appropriate measure to quantify the reversal of symptom(s), e.g., a visual analog scale for self-reporting, a heart rate monitor for tachycardia, etc. “Apparent” reversal of symptom(s) includes, but need not extend to, complete reversal.

As used herein, the term “cannabinoid” is synonymous with “cannabinoid receptor agonist” and refers to a compound which binds to and activates a cannabinoid receptor. The term includes both natural and synthetic compounds.

As used herein, the term “synthetic cannabinoid” (“SC”) means a non-naturally-occurring cannabinoid. While not synthetic analogues of THC and other naturally occurring cannabinoids, SCs share many common features with THC. Most are lipid-soluble, non-polar, small molecules (usually about 20-26 carbon atoms) that are fairly volatile, and often have a side-chain of 5-9 saturated carbon atoms which is associated with psychotropic activity from binding CB1 receptors. There are at least five major structural categories for synthetic cannabinoids: classical cannabinoids, non-classical cannabinoids, hybrid cannabinoids, aminoalkylindoles (and their analogues), and eicosanoids. Classical cannabinoids are analogs of THC that are based on a dibenzopyran ring; examples include nabilone, dronabinol, and the (−)-1,1-dimethylheptyl analog of 11-hydroxy-Δ8-tetrahydrocannabinol (HU-210). Non-classical cannabinoids include cyclohexylphenols such as cannabicyclohexanol. Hybrid cannabinoids have a combination of classical and non-classical cannabinoid structural features. Aminoalkylindoles are structurally dissimilar to THC and include naphthoylindoles such as 1-pentyl-3-(1-naphthoyl)indole (JWH-018), phenylacetylindoles such as 1-pentyl-3-(2-methoxyphenylacetyl)indole (JWH-250), and benzoylindoles such as 1-[(N-methylpiperidin-2-yl)methyl]-3-(2-iodobenzoyl)indole (AM-2233); they are the most common SCs found in SC blends due to relative ease of synthesis. Other compounds structurally similar to aminoalkylindoles include naphthoylpyrroles, naphthylmethylindenes, phenylacetylindoles/benzoylindoles, tetramethylcyclopropylindoles, adamantoylindoles, indazole carboxamides, indolecarboxylates, and quinolinyl esters. Eicosanoid SCs are analogs of endocannabinoids such as anandamide.

As used herein, the term “cannabinoid receptor antagonist” refers to a compound which binds to and blocks or dampens the normal biological function of the receptor and its signaling, especially in the presence of an agonist or partial agonist. The term includes cannabinoid receptor antagonists that are selective or nonselective for the CB1 receptor subtype, i.e., a “CB 1 antagonist.”

As used herein, the term “Cmax” refers to the maximum observed plasma concentration.

As used herein, the term “intramuscular (IM)” means into a muscle. Suitable muscles, if of sufficient mass, include the deltoid (upper arm), the thigh (esp. the anterolateral aspect of the thigh; particularly useful if via an autoinjector), the gluteus maximus (typically only adults and children>3 years old), and hip. The IM injection may be via a classical syringe or an autoinjector device.

As used herein, the term “intravenous (IV)” means delivered as a liquid into a vein of a patient. Intravenous administration can be by injection (in a relatively small volume and at relatively high concentration) by injection via a syringe or into a previously-inserted IV catheter, or by intravenous infusion (“IVN,” in a relatively larger and more dilute volume). IV administration, particularly injection, can be done in one or more pushes.

The terms “non-ionic solubilizer” and/or “emulsifying agent” and/or “solubilizing agent” are generally interchangeable as used herein, and include agents that result in formation of a micellar solution or a true solution of the agent being solubilized and a typically immiscible partner (for example, drinabant, which has a high logP, and water, which has a negative logP). Solubilizing agents include cationic and nonionic surfactants, and in certain circumstances may also act as absorption or permeation enhancers. One example of a solubilizing agent is Kolliphor HS 15/Solutol HS 15 (e.g., CAS No. 70142-34-6 or 61909-81-7, polyoxyl 15 hydroxystearate, polyglycol mono- and di-esters of 12-hydroxystearic acid with about 30% polyethylene glycol).

As used herein, the terms “overdose,” “intoxication,” and “poisoning” are synonymous and may be used interchangeably, and refer to the condition of having taken into the body of a subject, e.g. by inhalation or ingestion, an excess of a physiologically active and and/or psychoactive substance, such that the normal functioning of the body or one of its functions or parts is perturbed and the subject is at risk of harm.

As used herein, the term “parenteral” means administered by means other than oral, nasal (i.e., bypassing mucous membranes) or rectal intake, particularly intravenously or by injection elsewhere, e.g., intramuscular or subcutaneous injection.

As used herein, the term “push” in the context of an intravenous (IV) push is the rapid administration of a small volume of medication into a patient's vein, typically via a previously inserted IV catheter. Multiple pushes make be used to comprise a single IV dose.

As used herein, the term “subcutaneous” means “under the skin,” i.e., administered into the subcutis, the layer of skin directly below the dermis and epidermis (collectively referred to as the cutis), above muscle.

As used herein, a “symptom” of intoxication or overdose is a physical or mental feature that is regarded as is a departure from normal function or feeling. Common symptoms of cannabinoid intoxication or overdose include dry mouth, increased appetite, nystagmus, slurred speech, and conjunctival injection (red eye), as well as generalized psychomotor impairment, including impaired attention, reduced alertness (drowsiness), impaired concentration, slowed reaction time, impaired short-term memory, impaired executive functioning, and confusion. More serious symptoms may include postural/orthostatic hypotension, hypertension, tachycardia, nausea, delirium, agitation, anxiety, panic attacks, myoclonic jerking, sedation, paranoia, and hallucination. Severe effects may include seizures, hyperthermia, rhabdomyolysis, renal failure, angina, and myocardial infarction. Symptoms may present differently in children, and are known in the art. Symptoms of cannabinoid intoxication or overdose may be divided into cardiovascular symptom(s), neuropsychiatric symptom(s), and gastrointestinal symptom(s), with some overlap (e.g., a panic attack has both physical and neuropsychiatric components).

As used herein, the term “in need of treatment” and the term “in need thereof” when referring to treatment are used interchangeably and refer to a judgment made by a health caregiver (e.g. physician, nurse, nurse practitioner, that a patient will benefit from treatment.

As used herein, the term “subject” is intended to be synonymous with “patient,” and refers to any mammal (preferably human) who is intoxicated or overdosed with a cannabinoid.

### Cannabinoid Receptor Antagonists

Cannabinoid receptor antagonists are known in the art, and may be selective or nonselective for CB1. Potency and selectivity for the CB1 receptor are generally desirable because cannabinoids producing intoxication and overdose are CB1 agonists. Rimonabant (5-(4-chlorophenyl)-1-(2,4-dichloro-phenyl)-4-methyl-N-(piperidin-1-yl)-1H-pyrazole-3-carboxamide) was the first developed potent and selective CB1 antagonist, once approved as an anti-obesity agent in Europe. Many CB1 antagonists reported so far are 1,5-diarylpyrazole analogs, often featuring a para-substituted phenyl ring at the pyrazole 5-position and a 2,4-dichloro-substituted phenyl ring at the pyrazole 1-position. Other analogues in this class include surinabant and AM-251. Additional analogues featuring core replacements include 3,4-diarylpyrazolines, 4,5- diarylimidazoles, and 1,5-diarylpyrrole-3-carboxamides, fused bicyclic derivatives of diaryl- pyrazole, diaryl-imidazole, diaryl-purines (e.g. otenabant), six-membered ring pyrazole bioisosteres such as 2,3-diarylpyridines and 2,3-diarylpyrimidines, and methylsulfonamide azetidine derivatives. This last class includes drinabant ((±)-N-11-[bis(4-chlorophenyl)methyl]- 3-azetidinyl}-N-(3,5-difluorophenyl)methanesulfonamide), also potent and selective for CB1. Rimonabant, once approved as an oral formulation for the treatment of obesity and smoking cessation in Europe, was withdrawn from the European market, and the development of drinabant halted, due to rimonabant's concerning side effects including psychiatric effects such as depression and mood alterations.

However, these side effects are of far less concern when the intended use, instead of daily and long-term, will be limited to indications where the administration is acute and transient, such as reversal of cannabinoid overdose. Accordingly, any potent and selective CB1 antagonist with adequate target tissue penetration would be expected to work in the methods and formulations disclosed herein. Drinabant is preferred in certain embodiments, for example where reduction in tachycardia is important.

Plasma concentrations of CB1 antagonists useful in reversing cannabinoid overdose or one or more symptoms thereof will vary based on several factors, including the identity of the antagonist. For example, plasma concentrations of drinabant useful in reversing cannabinoid overdose or one or more symptoms thereof range from about 200 ng/mL to about 730 ng/mL. Additionally, it is understood by those skilled in the art that the because drinabant has been characterized as a competitive CB1 receptor antagonist, effective (therapeutic) plasma concentrations are dependent upon the dose and type of cannabinoid (a SC, THC, or a combination thereof) responsible for the overdose. Both the onset and degree of symptom relief may vary, and some symptoms (e.g. cardiovascular symptoms such as tachycardia) may be more sensitive to reversal than others (e.g., alertness, as measured by a clinician's impression and/or assessment through a visual analog scale (VAS)). Onset of symptom relief should be apparent within about 5 min to about 30 min following intravenous administration and about 15 min to about 45 min following intramuscular or subcutaneous administration, respectively.

In order to achieve these plasma concentrations, intravenous doses of drinabant of between about 1 mg and about 150 mg, about 1 mg and about 100 mg, or between about 1 mg and about 60 mg, or between about 15 mg and about 60 mg, or between about 30 mg and about 60 mg, or between about 1 mg and about 30 mg, or between about 15 mg and about 30 mg, or between about 50 mg and about 100 mg may be administered. Intravenous doses can be injected in volumes of about 1 to about 20 mL (lower volumes are preferred in certain circumstances).

Alternatively, in order to achieve these plasma concentrations, intramuscular or subcutaneous doses of drinabant of between about 1 mg and about 150 mg, about 5 mg and about 100 mg, or between about 5 mg and about 60 mg, or between about 15 mg and about 60 mg, or between about 30 mg and about 60 mg, or between about 15 mg and about 30 mg, or between about 50 mg and about 100 mg, or between about 5 mg and about 50 mg, or between about 5 mg and about 30 mg. Intramuscular or subcutaneous doses can be injected in a volume of up to about 2.5 mL for IM and about 1.5 mL for SC. Intramuscular injections are typically into a deltoid or gluteal muscle.

In certain embodiments, the doses above take into account the possibility that fatty tissue may act as a sink for a lipophilic cannabinoid antagonist, which may diffuse in and out of the fatty tissue over time. In any event, if no response is observed with ˜30-45 minutes of parenteral administration, and the presence of cannabinoids is confirmed or strongly suspected, a second dose may be administered.

### EXAMPLES

Table 1 below discloses several examples of compositions which can be formulated for parenteral administration (i.e., as liquid preparations) comprising an amount of drinabant or a salt or polymorph thereof effective to reverse cannabinoid overdose or one or more symptoms thereof in a subject.

TABLE 1

| Ex. | Mode | Dose, mg | Vol, mL |
|---|---|---|---|
| 1 | IV | 1 | 1 |
| 2 | IV | 1 | 2 |
| 3 | IV | 1 | 3 |
| 4 | IV | 1 | 5 |
| 5 | IV | 1 | 10 |
| 6 | IV | 5 | 1 |
| 7 | IV | 5 | 2 |
| 8 | IV | 5 | 3 |
| 9 | IV | 5 | 5 |
| 10 | IV | 5 | 10 |
| 11 | IV | 15 | 1 |
| 12 | IV | 15 | 2 |
| 13 | IV | 15 | 3 |
| 14 | IV | 15 | 5 |
| 15 | IV | 15 | 10 |
| 16 | IV | 30 | 1 |
| 17 | IV | 30 | 2 |
| 18 | IV | 30 | 3 |
| 19 | IV | 30 | 5 |
| 20 | IV | 30 | 10 |
| 21 | IV | 45 | 1 |
| 22 | IV | 45 | 2 |
| 23 | IV | 45 | 3 |
| 24 | IV | 45 | 5 |
| 25 | IV | 45 | 10 |
| 26 | IV | 60 | 1 |
| 27 | IV | 60 | 2 |
| 28 | IV | 60 | 3 |
| 29 | IV | 60 | 5 |
| 30 | IV | 60 | 10 |
| 31 | IV | 100 | 1 |
| 32 | IV | 100 | 2 |
| 33 | IV | 100 | 3 |
| 34 | IV | 100 | 5 |
| 35 | IV | 100 | 10 |
| 36 | IM | 5 | 1 |
| 37 | IM | 5 | 1.5 |
| 38 | IM | 5 | 2 |
| 39 | IM | 5 | 2.5 |
| 40 | IM | 15 | 1 |
| 41 | IM | 15 | 1.5 |
| 42 | IM | 15 | 2 |
| 43 | IM | 15 | 2.5 |
| 44 | IM | 30 | 1 |
| 45 | IM | 30 | 1.5 |
| 46 | IM | 30 | 2 |
| 47 | IM | 30 | 2.5 |
| 48 | IM | 45 | 1 |
| 49 | IM | 45 | 1.5 |
| 50 | IM | 45 | 2 |
| 51 | IM | 45 | 2.5 |
| 52 | IM | 60 | 1 |
| 53 | IM | 60 | 1.5 |
| 54 | IM | 60 | 2 |
| 55 | IM | 60 | 2.5 |
| 56 | IM | 100 | 1 |
| 57 | IM | 100 | 1.5 |
| 58 | IM | 100 | 2 |
| 59 | IM | 100 | 2.5 |
| 60 | SC | 5 | 0.5 |
| 61 | SC | 5 | 1 |
| 62 | SC | 5 | 1.5 |
| 63 | SC | 15 | 0.5 |
| 64 | SC | 15 | 1 |
| 65 | SC | 15 | 1.5 |
| 66 | SC | 30 | 0.5 |
| 67 | SC | 30 | 1 |
| 68 | SC | 30 | 1.5 |
| 69 | SC | 45 | 0.5 |
| 70 | SC | 45 | 1 |
| 71 | SC | 45 | 1.5 |
| 72 | SC | 60 | 0.5 |
| 73 | SC | 60 | 1 |
| 74 | SC | 60 | 1.5 |
| 75 | SC | 100 | 0.5 |
| 76 | SC | 100 | 1 |
| 77 | SC | 100 | 1.5 |

Table 2 below discloses several examples of compositions which can be formulated for parenteral administration as an IV infusion, comprising an amount of drinabant or a salt or polymorph thereof effective to reverse cannabinoid overdose or one or more symptoms thereof in a subject. The drinabant mat be delivered, e.g., over the given time interval (period, in minutes) below.

TABLE 2

| Ex. | Mode | Dose, mg | Vol., mL | Per., min |
|---|---|---|---|---|
| 78 | IVN | 5 | 50 | 30 |
| 79 | IVN | 5 | 100 | 30 |
| 80 | IVN | 5 | 125 | 30 |
| 81 | IVN | 15 | 50 | 30 |
| 82 | IVN | 15 | 100 | 30 |
| 83 | IVN | 15 | 125 | 30 |
| 84 | IVN | 30 | 125 | 60 |
| 85 | IVN | 30 | 125 | 90 |
| 86 | IVN | 30 | 125 | 120 |
| 87 | IVN | 30 | 250 | 60 |
| 88 | IVN | 30 | 250 | 90 |
| 89 | IVN | 30 | 250 | 120 |
| 90 | IVN | 45 | 250 | 60 |
| 91 | IVN | 45 | 250 | 90 |
| 92 | IVN | 45 | 250 | 120 |
| 93 | IVN | 45 | 500 | 60 |
| 94 | IVN | 45 | 500 | 90 |
| 95 | IVN | 45 | 500 | 120 |
| 96 | IVN | 60 | 250 | 60 |
| 97 | IVN | 60 | 250 | 90 |
| 98 | IVN | 60 | 250 | 120 |
| 99 | IVN | 60 | 500 | 60 |
| 100 | IVN | 60 | 500 | 90 |
| 101 | IVN | 60 | 500 | 120 |

### Assays and Protocols

### Clinical Pharmacology Protocol for Cannabinoid Overdose

Pharmacokinetics. For determination of the concentration of plasma drinabant, venous blood may be collected in, e.g., heparinized polypropylene tubes (lithium heparin) of 4 mL. Blood samples may be taken at baseline and (by way of example only) 2.5, 5, 10, 15, 20, 30, and/or 45 min and 1, 1.5, 2, 3, 4, 5, 6, 12 and 24 h after oral administration of drinabant or matching placebo. After blood collection, the tubes are centrifuged within 30 min for 15 min at 2000 g at 4° C. Plasma samples may be stored at a temperature of ˜20° C.

Turbulent Flow Chromatography-Mass Spectrometry/Mass Spectrometry (TFC- MS/MS) is a validated method to analyze plasma drinabant concentrations. See, e.g., Zuurman, et al., 2010. Validation of this method included evaluation of selectivity for drinabant. In each run, standards (known amount of drinabant) man be included periodically (e.g., after every 10 samples). The limit of quantification has been reported in the art 0.2 ng/mL; the intra-assay coefficient of variation between 1.0 and 5.4%; the inter-assay coefficient of variation between 2.0 and 6.5%. Preferably, all blood samples are handled and analyzed according to GCP/GLP. Drinabant plasma pharmacokinetic parameters (including tₘₐₓ, Cₘₐₓ, AUC₀₋₂₄, AUC₀₋₂₄, AUC_{inf}t_{1/2}) may be determined using non-compartmental analysis from individual plasma concentration-time profiles.

Plasma concentrations of cannabinoids may be assessed by methods known in the art, e.g. as disclosed in Sorensen L K and Hasselstrøm J B, Sensitive Determination of Cannabinoids in Whole Blood by LC-MS-MS After Rapid Removal of Phospholipids by Filtration, J Anal Toxicol 2017 Jun. 1; 41(5):382-391.

Pharmacodynamics. Pharmacodynamic endpoints of the efficacy of various formulations and doses of cannabinoid antagonists such as drinabant in reversing cannabinoid overdose or one or more symptoms thereof can be measured using a variety of measures, including both objectively observable and subject-reported phenomena.

Objectively observable measures of symptoms of cannabinoid overdose include tachycardia and hypertension. Cannabis and other cannabinoids dose-dependently increase heart rate and blood pressure, and these symptoms can become life-threatening in certain subjects. Drinabant is expected to reduce tachycardia (i.e., reduce heart rate) and blood pressure in a subject intoxicated or overdosed with one or more cannabinoids.

Other objectively observable measures of symptoms of cannabinoid overdose include postural instability, measurable by methods known in the art. See, e.g., Browne JE and O'Hare N.J., Review of the Different Methods for Assessing Standing Balance, Physiotherapy 2001 87(9):489-495. Cannabinoids tend to increase postural instability, and a cannabinoid antagonist such as drinabant would be expected to reverse this effect. Drinabant is expected to reduce postural instability in a subject intoxicated or overdosed with one or more cannabinoids.

Two of the most frequently used subject-reported scales are visual analogue scales (VASs) in clinical (pharmacologic) research to measure subjective effects: VAS Bond and Lader (alertness, calmness and mood) and VAS Bowdle (psychedelic effects). See, e.g., Kleinloog D et al., Profiling the subjective effects of A⁹-tetrahydrocannabinol using visual analogue scales, Int J Methods Psychiatr Res, 2014 June; 23(2):245-56. Three separate clusters may be monitored that describe the spectrum of subjective effects of cannabinoids, including the perception VAS (“time”, “thoughts” and “high”), the relaxation VAS (“drowsy”, “muzzy”, “mentally slow” and “dreamy”) and dysphoria VAS (“voices”, “meaning” and “suspicious”). The effects of cannabinoids on these VASs or any subsets thereof may be measured, and the effects of cannabinoid antagonists measured as well by the decline in these measures after administering an amount of a cannabinoid antagonist, such as drinabant, therapeutically effective to reverse cannabinoid intoxication. Drinabant is expected to reverse measures of cannabinoid intoxication and overdose.

### Solubility Protocol

Drinabant may be added to a fixed volume of aqueous solution with and without various amounts of water soluble carriers such as Solutol HS 15 in screw capped bottles. Samples are shaken (alternatively, stirred) for a length of time (e.g., 48 hours) at room temperature, pH optionally adjusted, and any suspensions filtered through, e.g., a Whatman filter paper no 1. Filtered solutions are then analyzed for drinabant concentration using an appropriate method such as UV/visible spectrophotometry at an appropriate wavelength (nm) or by HPLC. It is expected that at low concentrations of solubilizing agent (e.g., 1, 5, or 10%), improvement in solubility will increase linearly, but that at higher concentrations this trend may deviate.

Although the present invention has been described with reference to specific details of certain embodiments thereof in the above examples, it will be understood that modifications and variations are encompassed within the spirit and scope of the invention.

What is claimed is:

1. A composition formulated for parenteral administration comprising an amount of drinabant or a salt or polymorph thereof effective to reverse cannabinoid overdose, or one or more symptoms thereof in a subject.

2. A method of reversing one or more symptoms cannabinoid overdose comprising parenterally administering the CB-1 antagonist drinabant or a salt or polymorph thereof.

3. The composition as recited in claim 1 or the method as recited in claim 2, wherein the plasma concentrations of achieved by parenteral administration of drinabant or salt or polymorph thereof sufficient to reverse the cannabinoid overdose symptom(s) is 200 to about 730 ng/ml.

4. The composition or method as recited in claim 3, wherein the parenteral route of administration is chosen from among intravenous (IV), intramuscular (IM), and subcutaneous (SC).

5. The composition or method as recited in claim 4, wherein the parenteral route of administration is IV.

6. The composition or method as recited in claim 5, wherein the amount of drinabant, or a salt or polymorph thereof, sufficient to treat, reverse, or reduce the CHS or one or more symptoms thereof is between about 1 mg and about 60 mg per intravenous dose.

7. The composition or method as recited in claim 6, wherein the amount of drinabant, or a salt or polymorph thereof sufficient to treat, reverse, or reduce the CHS or one or more symptoms thereof is between about 30 and about 60 mg per intravenous dose.

8. The composition or method as recited in claim 5, wherein the IV dose is delivered by IV injection.

9. The composition or method as recited in claim 8, wherein the IV dose is delivered in a liquid volume of between about 1 and about 20 mL.

10. The composition or method as recited in claim 5, wherein the IV dose is delivered by IV infusion.

11. The composition or method as recited in claim 10, wherein the IV infusion is delivered in a liquid volume of between about 125 to about 500 mL.

12. The composition or method as recited in claim 11, wherein the IV infusion is delivered over a period of about 1 hour to about 2 hours.

13. The composition or method as recited in claim 11 or claim 12, wherein the IV infusion is delivered at a rate of about 0.5 mL/min to about 2 mL/min.

14. The composition or method as recited in claim 4, wherein the parenteral route of administration is IM or SC.

15. The composition or method as recited in claim 14, wherein the amount of drinabant, or a salt or polymorph thereof sufficient to reverse the cannabinoid overdose symptom(s) is between about 5 and about 60 mg per IM or SC dose .

16. The composition or method as recited in claim 15, wherein the amount of drinabant, or a salt or polymorph thereof sufficient to reverse the cannabinoid overdose symptom(s) is between about 5 and about 30 mg per IM or SC dose .

17. The composition or method as recited in claim 15 or claim 16, wherein the intramuscular dose is delivered in a liquid volume of up to about 2.5 ml.

18. The composition or method as recited in claim 16, wherein the intramuscular dose is delivered in a liquid volume of about 1 to about 2.5 ml.

19. The composition or method as recited in claim 15 or claim 16, wherein the SC dose is delivered in a liquid volume of up to about 1.5 ml.

20. The composition or method as recited in claim 19, wherein the SC dose is delivered in a liquid volume of about 1 mL to about 1.5 ml.

21. The composition or method as recited in claim 4, wherein the IV, IVN, IM, or SC dose or injection contains at least one agent that acts as a non-ionic solubilizer and/or emulsifying agent.

22. The composition or method as recited in claim 1, wherein the symptom(s) of cannabinoid overdose is/are chosen from cardiovascular symptom(s), neuropsychiatric symptom(s), and gastrointestinal symptom(s).

23. The composition or method as recited in claim 22, wherein the cardiovascular symptom(s) is/are chosen from hypertension and tachycardia.

24. The composition or method as recited in claim 22, wherein the neuropsychiatric symptom(s) is/are chosen from agitation, confusion, drowsiness/lack of alertness, hallucinations, and feeling “high.”

25. The composition or method as recited in claim 22, wherein the gastrointestinal symptom(s) is/are chosen from nausea and vomiting.

26. The composition or method as recited in claim 1, wherein the onset of reversal of symptom(s) of cannabinoid overdose are apparent within 5-30 minutes following intravenous injection of drinabant, or a salt or polymorph thereof.

27. The composition or method as recited in any of claim 1, wherein the onset of reversal of symptom(s) of cannabinoid overdose are apparent within 15-45 min following intramuscular administration of drinabant, or a salt or polymorph thereof.

28. The method as recited in claim 2, wherein if no response is observed within 30-120 minutes of a first administration of drinabant, and the presence of cannabinoids is confirmed or strongly suspected, a second dose may be administered.

29. The method as recited in claim 2, wherein if no response is observed within 30-45 minutes of a first administration of drinabant, and the presence of cannabinoids is confirmed or strongly suspected, a second dose may be administered.
